(19) United States
(12) Patent Application Publication
(10) Pub. No.: US 2006/0094999 A1
(43) Pub. Date: May 4, 2006

(54) Apparatus for and method of diagnosing and treating patello-femoral misalignment

(76) Inventor: Dean E. Cropper, Ashland, OR (US)

Correspondence Address: Ronald Clay Harris, Jr., 2830 South Meade Street, Arlington, VA, 22206, US

(21) Appl. No.: 10/976,866

(22) Filed: Nov. 1, 2004

(51) Int. Cl. A61F 13/00; A61F 13/06

(52) U.S. Cl. 602/60; 602/62; 602/63

(57) ABSTRACT

An apparatus for, and method of, diagnosing and treating patello-femoral misalignment, which includes an inward tracking member that operatively fits over, and provides direct inward pressure against, a patella; wherein the inward tracking member provides a compressive force against the patella, thereby increasing the contact surface area between the patellofemoral articular tissue and an associated femoral trochlear groove; is disclosed.

### FIELD OF THE INVENTION

The present invention relates to an apparatus for, and a method of, diagnosing and treating patello-femoral misalignment. More particularly, the present invention relates to an apparatus and method that provide inward tracking to a patella, to increase function and speed rehabilitation.

### BACKGROUND OF THE INVENTION

The patellofemoral joint of the knee is an articulating joint between the patella and the femur. More specifically, this joint consists of an articular surface on the posterior of the patella and a corresponding articular surface on the anterior distal portion of the femur, also termed the trochlear groove. The posterior of the patella is contoured as a ridge, while the trochlea is contoured as a groove that is dimensioned to receive the patellar ridge in a complementary manner. Proper dynamic function of the patellofemoral joint requires that the patellar ridge accurately track the underlying trochlear groove when the knee is moved through flexion or extension.

Joint disorders nevertheless arise with varying severity, pain and dysfunction. Some less severe, albeit still painful, disorders involve minimal or no errors in patellar tracking of the trochlear groove. Other more serious disorders are characterized by patellar misalignment, i.e., transient displacement, or dislocation, i.e., permanent displacement, of the patella from the trochlear groove.

During functional movement of the knee joint, that is—typically from 0° to 60° of knee flexion, various tracking errors can occur due to injury, overuse, or changes inherent to adolescent growth, which in each of these cases causes pain and dysfunction. When the patella is seated in the trochlear groove at a range greater than about 60° of knee flexion, there is very little movement of the patella outside of the trochlear groove. Between about 0° and 40° of knee flexion, and especially about 20° to 40°, however, there is a propensity for the patella to track away from the groove as the knee flexes.

Rehabilitation of the weakened joint is often limited to the extent that correct tracking is absent, or ineffectively applied, and the resulting pain makes exercise too unbearable. As the quadriceps contract, they apply a lever force to the patellofemoral joint that is, more or less, directly related to the overall patellofemoral joint stress. Pain associated with such stress increases in relation to the amount of overall stress. Accordingly, as the quadriceps contract more powerfully, such as while going up stairs or doing squats, overall stress and associated pain increases.

In addition, patellofemoral joint stress at any given contact area increases as the overall patellofemoral joint stress is focused about a smaller patellofemoral contact surface area. Pain associated with such stress increases in direct relation to a reduction in the amount of patella-to-femur contact surface area. Thus, patellofemoral pain is not only directly related to the overall joint force applied between the patella and the femur; it is inversely related to the amount of patellofemoral contact surface area. Rehabilitation of the weakened joint through quadriceps contraction is therefore limited by the pain associated with both overall patellofemoral joint stress and a minimal patellofemoral contact surface area.

Because misalignment frequently occurs even early on in the extensor motion, it is moreover important to account for the damage and pain caused by both overall joint stress and a minimal patellofemoral contact surface area, throughout a full range of knee flexion and extension motion. If corrective tracking forces are effectively applied in conjunction with exercise to correct the relative patella-to-femur positioning problem for improved tracking, pain associated with low patella-to-femur contact surface area may be abated.

Prior attempts to provide patellofemoral support nevertheless fail to account for patellofemoral contact surface area as a factor in joint stress. For example, U.S. Pat. No. 6,287,269 B1, entitled “Dynamic Orthesis Device for the Conservative Treatment of Patellofemoral Instability of the Knee”, discloses a support element that provides medial tracking of the patella. It does not account for damage and pain caused by minimal patellofemoral contact surface area, however.

U.S. Pat. Nos. 6,077,242 and 6,080,124, respectively entitled “Patella Strap” and “Patella Strap Method”, disclose a strap that directs either superior or inferior, i.e., downward or upward, pressure on a patella. It does not account for damage and pain caused by a minimal patellofemoral contact surface area, however.

U.S. Pat. No. 6,592,539 B1, entitled “Orthotic or Prosthetic Sleeve Formed of Elasticized Fabric Sections Having Different Elastic Stiffness”, discloses a compression sleeve having a section that fits over the entire knee and includes unidirectional stretchable fabric that stretches along the sleeve's axis. It does not account for damage and pain caused by a minimal patellofemoral contact surface area, however.

U.S. Pat. No. 4,607,628, entitled “Patella Support Brace”, discloses a patella pad that exerts a medial and distal pressure on the patella during knee extension. It does not account for damage and pain caused by minimal patellofemoral contact surface area, however.

U.S. Pat. No. 6,551,264 B1, entitled “Orthosis for Dynamically Stabilizing the Patello-femoral Joint”, discloses a knee orthosis having a patellar tracking guide that tensions as the knee extends. It does not account for damage and pain caused by a minimal patellofemoral contact surface area, however.

All of these prior attempts also fail to provide an apparatus for, or method of, determining when damage and pain are at least in part caused by a minimal patellofemoral contact surface area.

Hence, the prior art fails to provide an apparatus for, or method of, diagnosis or treating pain caused by a minimal patellofemoral contact surface area throughout a full range of flexion and extension motion.

### SUMMARY OF THE INVENTION

Thus, the present invention is directed to an apparatus for diagnosing patello-femoral misalignment that provides direct inward pressure on a patella.

The present invention is also directed to a method of diagnosing patello-femoral misalignment that provides inward pressure on a patella throughout a full range of flexion and extension motion.

The present invention is also directed to an apparatus for treating patello-femoral misalignment that provides inward pressure on a patella.

The present invention is also directed to a method of treating patello-femoral misalignment that provides inward pressure on a patella throughout a full range of flexion and extension motion.

One aspect of the present invention is directed to an apparatus for diagnosing patello-femoral misalignment that comprises an inward tracking member that operatively fits over, and provides direct inward pressure against, a patella. The inward tracking member provides a compressive force against the patella, thereby increasing the contact surface area between the patellofemoral articular tissue and an associated femoral trochlear groove.

In another aspect the apparatus further comprises an elastic sleeve that operatively fits onto a knee, and has upper, middle, and lower portions, each portion having an internal diameter, wherein the effective internal diameter of the middle portion is less than the internal diameters of the upper and lower portions when the sleeve is not being worn.

In still another aspect, the internal diameter of the upper portion is greater than the internal diameter of the lower portion.

In yet another aspect, the sleeve is made of breathable, multi-directional-stretch fabric.

In still another aspect, the tracking member runs horizontally across the patella from one side of the apparatus to another.

In yet another aspect, the compressive force against the patella is counterbalanced by a force that is distributed along a length of the apparatus that is longer than a width of the inward tracking member.

In still another aspect, the distributed force is distributed along a portion of the back and sides of the leg.

In yet another aspect, the distributed force is distributed through a semi-rigid side member.

In still another aspect, a side member runs along a length of an elastic sleeve.

In yet another aspect, the apparatus further comprises an elastic sleeve having a front member that has top, middle, and bottom portions each having horizontal widths, wherein a horizontal length of the inward tracking member is shorter than the width of the middle portion.

In still another aspect, two semi-rigid side members are respectively attached along two opposing sides of the front member.

In yet another aspect, the tracking member has an indicium that indicates a correct placement of the apparatus over the patella.

In still another aspect, a tensioning force from a side portion and a back side is concentrated onto the patella through the tracking member. the tracking member is sewn onto a side member.

In yet another aspect, the tracking member is operatively buckled onto, or fed through a loop on, a side member.

In still another aspect, the tracking member runs vertically across the patella from an upper portion to a lower portion of the apparatus, and the compressive force against the patella is counterbalanced by a force that is distributed to the upper and lower portions.

In yet another aspect, the upper and lower portions are connected by a side portion and separated by a length that is longer than the length of the tracking member.

In still another aspect, the upper and lower portions respectively fit around a user's upper and lower leg.

In yet another aspect, a length across which the force is distributed is longer than an effective operative length of the inward tracking member.

In still another aspect, the tracking member is an elastic strap that operatively provides adjustable tension across the patella.

In yet another aspect, the strap has indicia that indicate various levels of tension.

Another aspect of the invention is directed to an apparatus for treating patello-femoral misalignment that comprises an inward tracking member that operatively fits over, and provides direct inward pressure against, a patella. The inward tracking member provides a compressive force against the patella, thereby increasing the contact surface area between the patellofemoral articular tissue and an associated femoral trochlear groove.

Another aspect of the invention is directed to a method of diagnosing patello-femoral misalignment that comprises providing an apparatus that has an inward tracking member that operatively fits over, and provides direct inward pressure against, a patella; wherein the inward tracking member provides a compressive force against the patella, thereby increasing the contact surface area between the patellofemoral articular tissue and an associated femoral trochlear groove; instructing a user to apply a load to an injured knee; assessing an amount of pain experienced while loading the knee without the apparatus; instructing the user to don the apparatus and apply a load to the knee; and determining the relative pain experienced during load application with, and without, the apparatus.

In yet another aspect, the load is applied as the user steps up to an elevated surface.

Another aspect of the invention is directed to an apparatus for diagnosing patello-femoral misalignment that comprises an inward tracking member that operatively fits over, and provides concentrated inward pressure against, a patella. The inward tracking member provides a compressive force against the patella, thereby increasing the contact surface area between the patellofemoral articular tissue and an associated femoral trochlear groove.

In another aspect, the tracking member works throughout a full range of flexion and extension motion.

### BRIEF DESCRIPTION OF THE DRAWINGS

In the accompanying drawings, which form a part of the specification and are to be read in conjunction therewith, and in which like reference numerals are used to indicate like parts in the various views:

FIG. 1 is a front perspective view of one embodiment of an apparatus according to the present invention;

FIG. 2 is a back perspective view of the apparatus of FIG. 1;

FIG. 3 is a side perspective view of the apparatus of FIG. 1 as it is worn and used in preparation for loading a knee joint in one embodiment of a method according to the present invention;

FIG. 4 is a side perspective view of the apparatus of FIG. 1 as it is worn and used while loading a knee joint in the method embodiment of FIG. 4;

FIG. 5 is a front perspective view of a second embodiment of an apparatus according to the present invention;

FIG. 6 is a side perspective view of the apparatus of FIG. 5 as it is worn and used to diagnose and/or treat a patello-femoral misalignment of a knee; and

FIG. 7 is a side perspective view of a third embodiment of an apparatus according to the present invention, as it is worn and used to diagnose and/or treat a patello-femoral misalignment in a second embodiment of a method according to the present invention

### DETAILED DESCRIPTION OF THE INVENTION

As illustrated in the accompanying drawings and discussed in detail below, one aspect of the present invention is directed to an apparatus for diagnosing patello-femoral misalignment that, when used, indicates whether a treatment of direct inward pressure onto the patella is appropriate. By comparing the relative pain experienced while wearing the apparatus during load application with, the pain experienced while not wearing it, a correct treatment is suggested. If knee pain is decreased when wearing the apparatus, then the apparatus, or another apparatus that also applies direct inward pressure onto the patella, is appropriate for treating the knee.

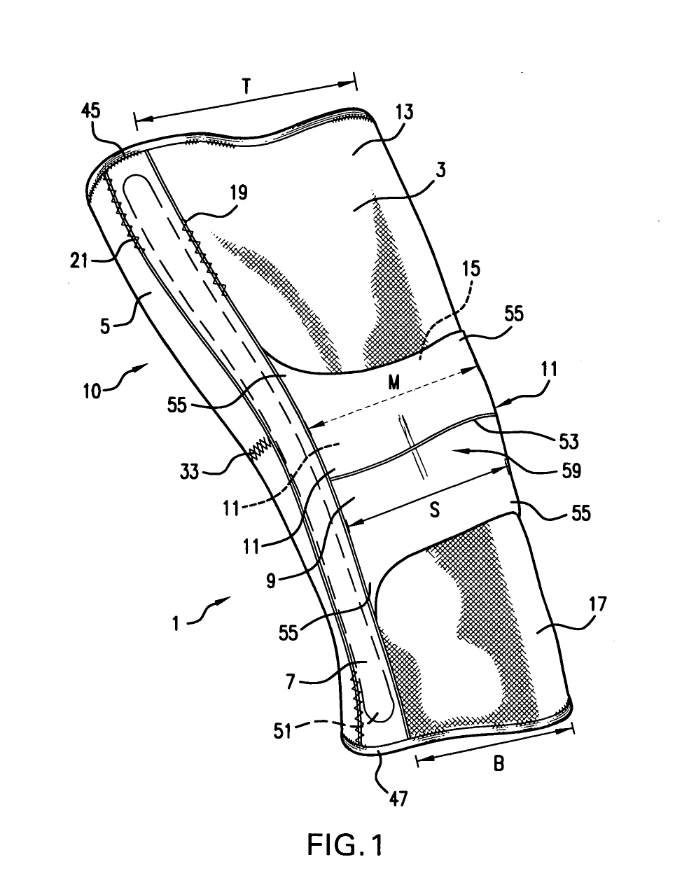

Referring to FIG. 1, patello-femoral diagnostic sleeve 1 includes front panel 3, back side 5, sides portions 7, and elastic inward tracking strap 9. Front panel 3 and back side 5 are sewn to left and right side portions 7 at respective front 19 and back 21 side seams to form elastic compression sleeve 1, which generally tapers in actual internal diameter as it runs from top to bottom. Front panel 3 has top section 13, middle section 15, and lower section 17, and it generally tapers in width as it runs from its top to its bottom. Thus, width T of top section 13 is longer than width M of middle section 15, which is longer than width B of lower section 17.

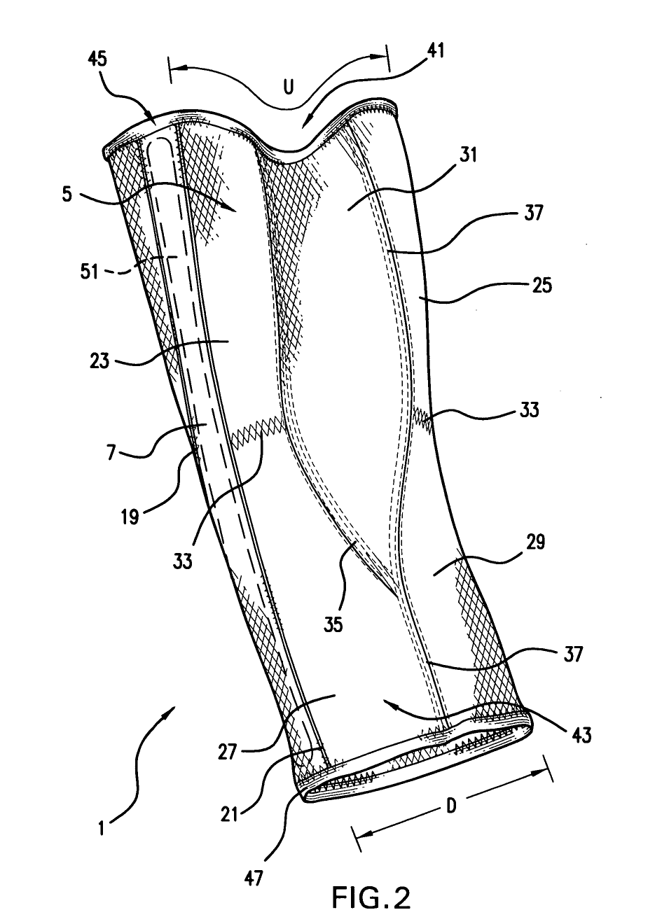

Referring to FIG. 2, back side 5 includes upper left 23 and right 25 back panels, lower left 27 and right 29 back panels, and center panel 31, which are sewn to one another at left and right horizontal seams 33, and left 35 and right 37 vertical seams. Back side 5 generally tapers in width as it runs from its top to its bottom. Thus, width U of upper portion 41 of back side 5 is wider than width D of lower portion 43 of back side 5. The top and bottom of elastic sleeve 1 are bordered by elastic fabric bands 45 and 47, which are made of an elastic fabric that stretches roughly to the same degree as the fabric used in the elastic panels of sleeve 1.

In one embodiment, sleeve 1 is constructed of an elastic, breathable, compressive synthetic multi-directional stretch material, such as is disclosed in U.S. Pat. No. 5,735,807, which is hereby incorporated by reference in its entirety. Any elastic, inelastic, synthetic or natural material suitable for wearing can be used to construct a sleeve according to this aspect of the present invention.

Side portions 7 contain semi-rigid plastic support slats 51, which run the length of sleeve 1. In particular, slats 51 are made from flexible aluminum flattened coiled wire or plastic such as nylon, but they can be made from any semi-rigid, flexible material that will allow a user to bend his or her knee while wearing the apparatus.

Referring again to FIG. 1, elastic inward tracking strap 9 is sewn to respective side portions 7 at its two ends 11 and horizontally spans middle section 15 of front panel 3. Length S of strap 9 is shorter than width M of middle section 15 when the sleeve is not being worn by a user. As a result, the effective internal diameter of sleeve 1 is less at its middle portion than at its top or bottom portions when sleeve 1 is not being worn by a user.

Strap 9 is made of a single layer elastic multi-directional stretch material, such as a nylon-Lycra® blend. Any elastic, inelastic, synthetic or natural material suitable for applying inward force onto a patella can be used to construct the strap of this aspect of the present invention. Any device or material that can provide direct force onto a patella is otherwise suitable for use in the present invention.

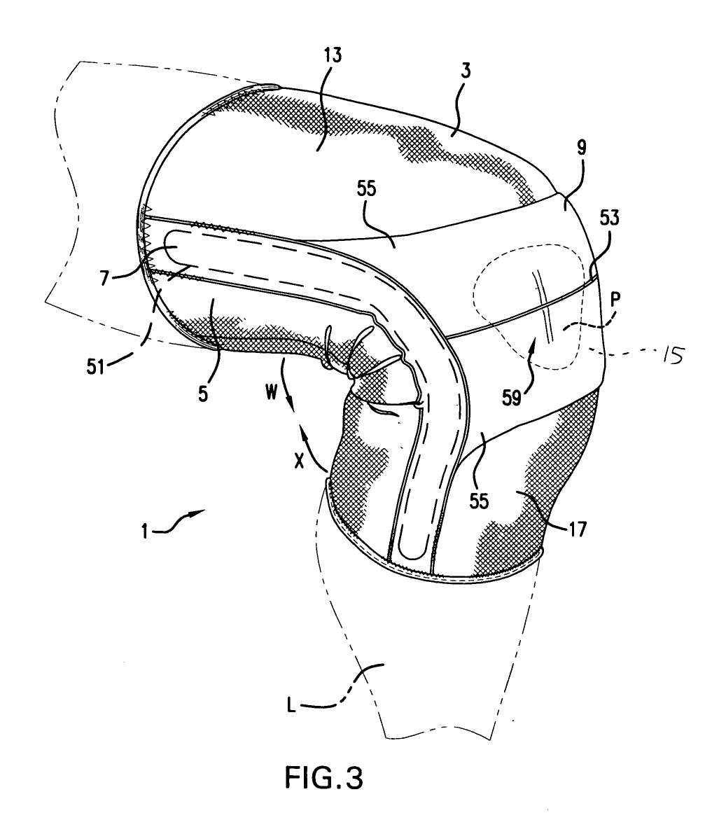

Referring to FIG. 3, when worn over a knee, middle portion 15 of sleeve 1, and strap 9 both stretch to form fit over patella P, but tension produced by the expansive stretching of sleeve 1 around leg L is directly concentrated onto patella P at center 59 of strap 9. Horizontal and vertical line indicium 53 on strap 9 indicates the vertical and horizontal centers of strap 9, and is intended to assist the user with centering strap 9 directly over the center of patella P.

After the user dons sleeve 1 such that indicium 53 lies directly over patella P, strap 9 is stretched and thereby provides compressive direct, inward force onto patella P throughout the entire range of knee flexion and extension motions. This stretching occurs as tension is applied at strap 9's ends by attached side portions 7 and back side 5 as sleeve 1 is forced to expand by leg L. The force against patella P by center 59 of strap 9 is applied by a length of side portions 7 that is longer than the width of strap 9. The counterbalancing force applied by the ends of strap 9 is distributed along the entire length of side portions 7 and back side 5, in particular through semi-rigid slats 51. Thus tensioning force from stretched side portions 7 and back side 5 is concentrated onto the front of sleeve 1 onto patella P through strap 9.

The width of strap 9 widens at its corners 55, which help to further distribute the counterbalancing force applied across the entire length of side portions 7 and around back side 5 of sleeve 1. As a result, sleeve 1 applies a direct inward compressive force against patella P that decreases pain by increasing the contact area between patella P and the user's associated trochlear groove. This increase in contact area tends to decrease otherwise sharp pain that results from a relatively small patella-to-femur contact area.

Therefore, if direct inward compressive force is an appropriate treatment for a user's knee, the pain experienced by a user wearing sleeve 1 while loading his knee will be less than the pain experienced by the same user who loads his knee while not wearing sleeve 1. Other embodiments of this aspect are described below.

A second aspect of the invention is accordingly directed to a method for diagnosing patello-femoral misalignment, in which the user loads his or her knee while wearing sleeve 1, and while not wearing sleeve 1. In one embodiment, a healthcare professional instructs the user to don sleeve 1 such that indicium 53 falls over the center of patella P, and apply a load to the knee. The user places his or her foot onto a raised surface such that his or her knee is initially in a flexed, i.e., bent, position. Such a movement occurs in accordance with direction arrows W and X.

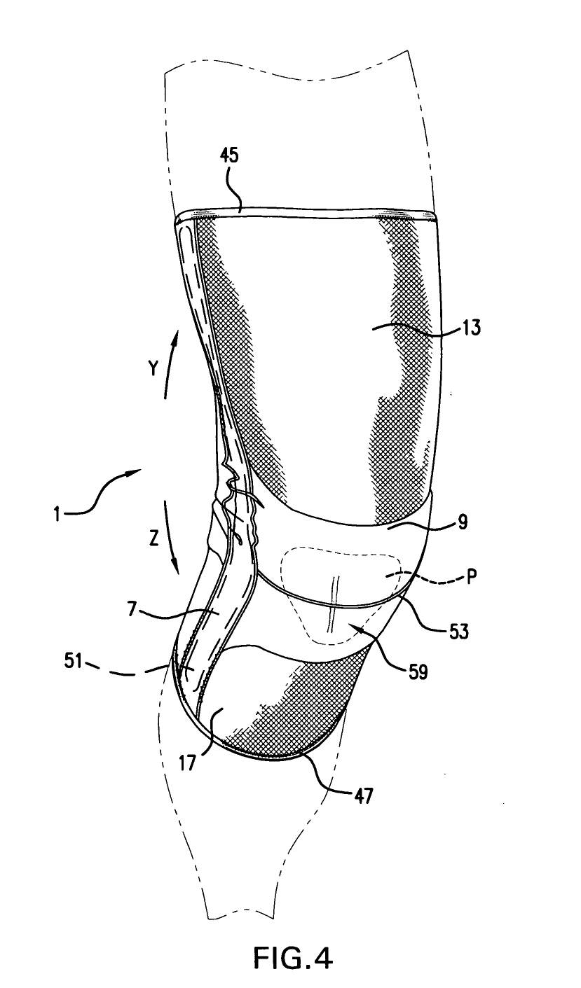

Referring to FIG. 4, the user then extends his or her knee by stepping up onto the raised surface, and thereby applies a load to his or her knee that is roughly three times the normal load experienced while standing. Such a movement occurs in accordance with directional arrows Y and Z. The same load is applied to the knee in the same fashion, but without wearing sleeve 1. Many other techniques to apply various loads to a knee can be used, however.

The relative pain levels are determined by having the user rate his pain on a scale from 1-10. A reduction in pain experienced by the user while wearing sleeve 1 indicates that the type of direct inward, compressive force afforded by sleeve 1 is an appropriate treatment. Any automated or manual technique—whether it be through electronic (or other) instrumentation readings or a user's own assessment of relative pain—can be used to comparatively determine such relative effectiveness of sleeve 1.

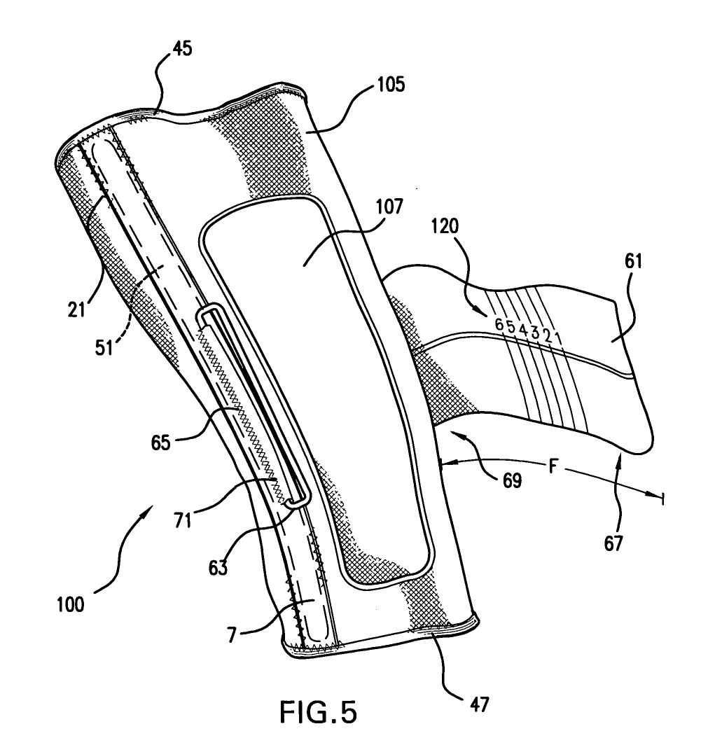

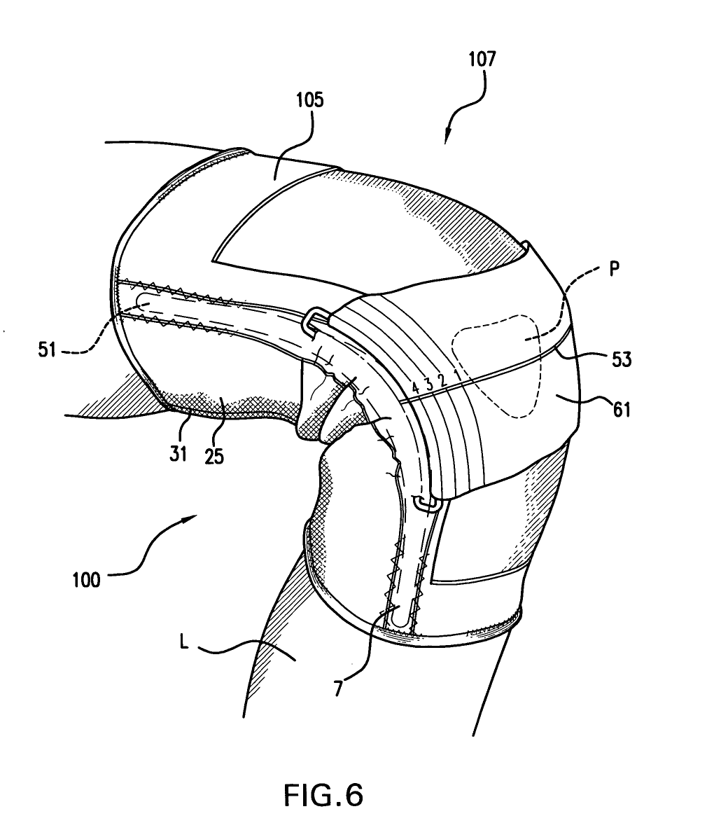

While several features of this aspect of the invention are described above, further additional embodiments include the use of various alternate embodiments of the apparatus. Referring to FIGS. 5 and 6, in one embodiment front panel 105, which has large opening 107, comprises sleeve 100.

Strap 61 feeds through flexible loop 63, which is sewn onto right side portion 7 with fabric 71 and stitching 65, tensioned, and secured back onto itself using hook 67 and loop 69 fabric. Strap 61 has a length F, which is considerably longer than length S (shown in FIG. 1). Strap 61 has vertical indicia 120, which include numbers and vertical lines that indicate relative tensioning of strap 9.

Loop 63 can be rigid or flexible. Any fastening device, such as a buckle, clasp, snap, or zipper can be used. Any device suitable for creating tension that applies pressure onto a patella can be used in this embodiment of the present invention.

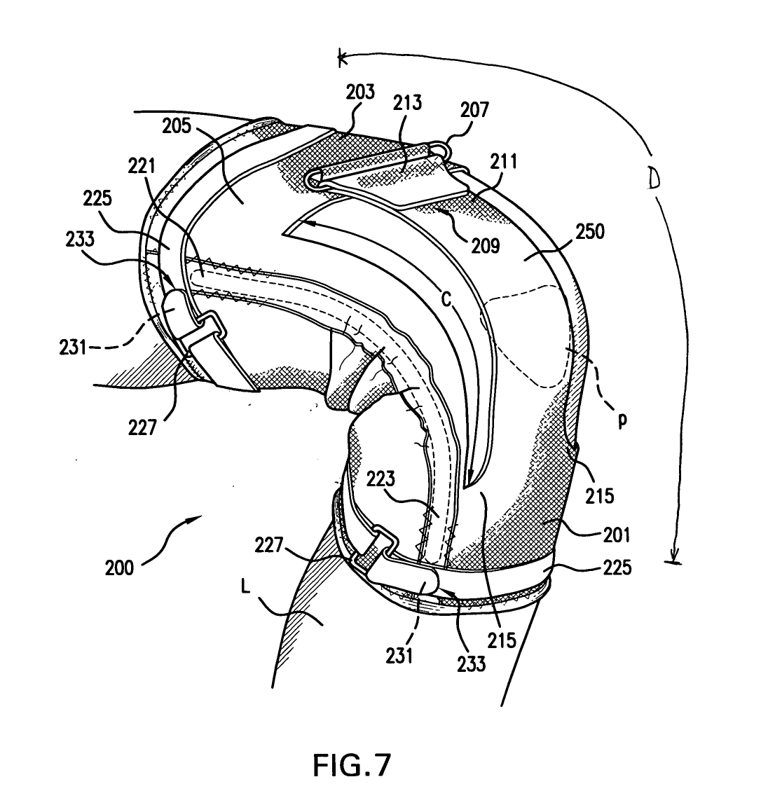

Referring to FIG. 7, sleeve 200 includes vertically tensioned elastic strap 250, which extends from lower portion 201 of front panel 203, to upper portion 205 of panel 203. Strap 250 operatively loops through loop 207 and is secured to itself using hook 209 and loop 211 fabric.

Strap 250 applies inward, concentrated force onto patella P throughout the entire range of flexion and extensor knee motion, as lower 201 and upper 205 portions, upper 221 and lower 223 ends of side portions 7, and back side 5 provide tension to strap 250.

Strap 250 applies opposing, counterbalancing force, which is distributed to lower 201 and upper 205 portions, upper 221 and lower 223 ends of side portions 7, and back side 5. Effective operative length C is shorter than length D of the sleeve to which such force is distributed. Corners 215 and widened end 213 of strap 250 further distribute such force throughout sleeve 1.

Belts 225 feed through loops and back onto themselves with hook 231 and loop 233 fabric to secure lower 201 and upper 203 portions of sleeve 200 to their respective vertical positions along leg L.

A third aspect of the present invention is directed to an apparatus for treating patello-femoral misalignment, several embodiments of which are substantially described above. Several additional features and embodiments of this aspect are disclosed in U.S. patent application Ser. No. 10/796,171, filed Mar. 10, 2004, which is hereby incorporated by reference in its entirety.

A fourth aspect of the present invention is directed to a method for treating patello-femoral misalignment that includes the application and intermittent tensioning of a suitable treatment device, such as one of the above-described embodiments of the present invention.

While it is apparent that the illustrative embodiments of the invention disclosed herein fulfill the objectives of the present invention, it is appreciated that numerous modifications and other embodiments may be devised by those skilled in the art. Additionally, feature(s) and/or element(s) from any embodiment may be used singly or in combination with other embodiment(s). Therefore, it will be understood that the appended claims are intended to cover all such modifications and embodiments that would come within the spirit and scope of the present invention.

I claim:

1. An apparatus for diagnosing patello-femoral misalignment, comprising:
an inward tracking member that operatively fits over, and provides direct inward pressure against, a patella;
wherein the inward tracking member provides a compressive force against the patella, thereby increasing the contact surface area between the patellofemoral articular tissue and an associated femoral trochlear groove.

2. The apparatus of claim 1 wherein the apparatus further comprises an elastic sleeve that operatively fits onto a knee, and has upper, middle, and lower portions, each portion having an internal diameter;
wherein the effective internal diameter of the middle portion is less than the internal diameters of the upper and lower portions when the sleeve is not being worn.

3. The apparatus of claim 2 wherein the internal diameter of the upper portion is greater than the internal diameter of the lower portion.

4. The apparatus of claim 1 wherein the sleeve is made of breathable, multi-directional-stretch fabric.

5. The apparatus of claim 1 wherein the tracking member runs horizontally across the patella from one side of the apparatus to another.

6. The apparatus of claim 1 wherein the compressive force against the patella is counterbalanced by a force that is distributed along a length of the apparatus that is longer than a width of the inward tracking member.

7. The apparatus of claim 6 wherein the distributed force is distributed along a portion of the back and sides of the leg.

8. The apparatus of claim 6 wherein the distributed force is distributed through a semi-rigid side member.

9. The apparatus of claim 1 wherein a side member runs along a length of an elastic sleeve.

10. The apparatus of claim 1 wherein the apparatus further comprises an elastic sleeve having a front member that has top, middle, and bottom portions each having horizontal widths, wherein a horizontal length of the inward tracking member is shorter than the width of the middle portion.

11. The apparatus of claim 10 wherein two semi-rigid side members are respectively attached along two opposing sides of the front member.

12. The apparatus of claim 1 wherein the tracking member has an indicium that indicates a correct placement of the apparatus over the patella.

13. The apparatus of claim 1 wherein a tensioning force from a side portion and a back side is concentrated onto the patella through the tracking member.

14. The apparatus of claim 1 wherein the tracking member is sewn onto a side member.

15. The apparatus of claim 1 wherein the tracking member is operatively buckled onto, or fed through a loop on, a side member.

16. The apparatus of claim 1 wherein the tracking member runs vertically across the patella from an upper portion to a lower portion of the apparatus, and the compressive force against the patella is counterbalanced by a force that is distributed to the upper and lower portions.

17. The apparatus of claim 16 wherein the upper and lower portions are connected by a side portion and separated by a length that is longer than the length of the tracking member.

18. The apparatus of claim 16 wherein the upper and lower portions respectively fit around a user's upper and lower leg.

19. The apparatus of claim 16 wherein a length across which the force is distributed is longer than an effective operative length of the inward tracking member.

20. The apparatus of claim 1 wherein the tracking member is an elastic strap that operatively provides adjustable tension across the patella.

21. The apparatus of claim 20 wherein the strap has indicia that indicate various levels of tension.

22. An apparatus for treating patello-femoral misalignment, comprising:
an inward tracking member that operatively fits over, and provides direct inward pressure against, a patella;
wherein the inward tracking member provides a compressive force against the patella, thereby increasing the contact surface area between the patellofemoral articular tissue and an associated femoral trochlear groove.

23. The apparatus of claim 22 wherein the apparatus further comprises an elastic sleeve that operatively fits onto a knee, and has upper, middle, and lower portions, each portion having an internal diameter;
wherein the effective internal diameter of the middle portion is less than the internal diameters of the upper and lower portions when the sleeve is not being worn.

24. The apparatus of claim 23 wherein the internal diameter of the upper portion is greater than the internal diameter of the lower portion when the sleeve is not being worn.

25. The apparatus of claim 22 wherein the sleeve is made of breathable, multi-directional-stretch fabric.

26. The apparatus of claim 22 wherein the tracking member runs horizontally across the patella from one side of the apparatus to another.

27. The apparatus of claim 22 wherein the compressive force against the patella is counterbalanced by a force that is distributed along a length of the apparatus that is longer than a width of the inward tracking member.

28. The apparatus of claim 27 wherein the distributed force is distributed along a portion of the back and sides of the leg.

29. The apparatus of claim 27 wherein the distributed force is distributed through a semi-rigid side member.

30. The apparatus of claim 22 wherein a side member runs along a length of an elastic sleeve.

31. The apparatus of claim 22 wherein the apparatus further comprises an elastic sleeve having a front member that has top, middle, and bottom portions each having horizontal widths, wherein a horizontal length of the inward tracking member is shorter than the width of the middle portion.

32. The apparatus of claim 31 wherein two semi-rigid side members are respectively attached along two opposing sides of the front member.

33. The apparatus of claim 32 wherein the tracking member has an indicium that indicates a correct placement of the apparatus over the patella.

34. The apparatus of claim 33 wherein a tensioning force from a side portion and a back side is concentrated onto the patella through the tracking member.

35. The apparatus of claim 22 wherein the tracking member is sewn onto a side member.

36. The apparatus of claim 22 wherein the tracking member is operatively buckled onto, or fed through a loop on, a side member.

37. The apparatus of claim 22 wherein the tracking member runs vertically across the patella from an upper portion to a lower portion of the apparatus, and the compressive force against the patella is counterbalanced by a force that is distributed to the upper and lower portions.

38. The apparatus of claim 37 wherein the upper and lower portions are connected by a side portion and separated by a length that is longer than the length of the tracking member.

39. The apparatus of claim 37 wherein the upper and lower portions respectively fit around a user's upper and lower leg.

40. The apparatus of claim 37 wherein a length across which the force is distributed is longer than an effective operative length of the inward tracking member.

41. The apparatus of claim 22 wherein the tracking member is an elastic strap that operatively provides adjustable tension across the patella.

42. The apparatus of claim 41 wherein the strap has indicia that indicate various levels of tension.

43. A method of diagnosing patello-femoral misalignment, comprising:
providing an apparatus that has an inward tracking member that operatively fits over, and provides direct inward pressure against, a patella; wherein the inward tracking member provides a compressive force against the patella, thereby increasing the contact surface area between the patellofemoral articular tissue and an associated femoral trochlear groove; and
instructing a user to apply a load to an injured knee;
assessing an amount of pain experienced while loading the knee without the apparatus;
instructing the user to don the apparatus and apply a load to the knee; and
determining the relative pain experienced during load application with, and without, the apparatus.

44. The method of claim 43 wherein the apparatus further comprises an elastic sleeve that operatively fits onto a knee, and has upper, middle, and lower portions, each portion having an internal diameter;
wherein the effective internal diameter of the middle portion is less than the internal diameters of the upper and lower portions when the sleeve is not being worn.

45. The method of claim 44 wherein the internal diameter of the upper portion is greater than the internal diameter of the lower portion when the sleeve is not being worn.

46. The method of claim 43 wherein the sleeve is made of breathable, multi-directional-stretch fabric.

47. The method of claim 43 wherein the tracking member runs horizontally across the patella from one side of the apparatus to another.

48. The method of claim 43 wherein the compressive force against the patella is counterbalanced by a force that is distributed along a length of the apparatus that is longer than a width of the inward tracking member.

49. The method of claim 48 wherein the distributed force is distributed along a portion of the back and sides of the leg.

50. The method of claim 48 wherein the distributed force is distributed through a semi-rigid side member.

51. The method of claim 43 wherein the load is applied as the user steps up to an elevated surface.

52. An apparatus for diagnosing patello-femoral misalignment, comprising:
an inward tracking member that operatively fits over, and provides concentrated inward pressure against, a patella;
wherein the inward tracking member provides a compressive force against the patella, thereby increasing the contact surface area between the patellofemoral articular tissue and an associated femoral trochlear groove.

53. The apparatus of claim 52 wherein the tracking member works throughout a full range of flexion and extension motion.
